(19) United States
(12) Patent Application Publication
(10) Pub. No.: US 2025/0271395 A1
(43) Pub. Date: Aug. 28, 2025

(54) Portable DNA Sequencing System Utilizing Graphene Sensors and DNA/Carbon Nanotube Hybrid Structures

(71) Applicant: Board of Trustees of the University of Arkansas, Little Rock, AR (US)

(72) Inventors: Steve Tung, Fayetteville, AR (US); Han Hu, Bentonville, AR (US); Jin-Woo Kim, Fayetteville, AR (US); Jeremy Meyer, Barling, AR (US)

(21) Appl. No.: 19/064,202

(22) Filed: Feb. 26, 2025

Related U.S. Application Data
(60) Provisional application No. 63/557,733, filed on Feb. 26, 2024.

(51) Int. Cl. G01N 27/447; C12Q 1/6869

(52) U.S. Cl. CPC G01N 27/44791; C12Q 1/6869; G01N 27/4473

(57) ABSTRACT

A method of sequencing DNA is disclosed. A single-stranded DNA molecule is wrapped around a carbon nanotube to form a DNA/carbon nanotube hybrid structure. The DNA/carbon nanotube hybrid structure is then moved across a nanofluidic chip. The nanofluidic chip includes graphene sensors and a nanochannel extending across the graphene sensors. The graphene sensors are positioned between opposing electrodes. A power source applies a constant voltage to the graphene sensors between the electrodes. The electrical current through the graphene sensors between the electrodes is measured as the DNA/carbon nanotube hybrid structure moves across the graphene sensors. The composition of the bases of the single-stranded DNA molecule is determined based on the measured electrical current.

### CROSS-REFERENCE TO RELATED APPLICATIONS

This application claims the benefit of U.S. Provisional Application No. 63/557,733, entitled “Portable DNA Sequencing System Utilizing Graphene Sensors and DNA/Carbon Nanotube Hybrid Structures” and filed on Feb. 26, 2024. The complete disclosure of said patent application is hereby incorporated by reference.

### STATEMENT REGARDING FEDERALLY SPONSORED RESEARCH OR DEVELOPMENT

Not applicable

### BACKGROUND OF THE INVENTION

DNA sequencing has become an essential analytical tool for a wide range of biomedical research and clinical applications. According to a September 2022 report by SkyQuest Technology Consulting, the global sequencing market was valued at $5B in 2021 and expected to reach $30B by 2028. The current commercial sequencing market is dominated by major corporations such as Illumina and PacBio which utilize massively parallel sequencing platforms to conduct a sequencing protocol known as “Sequencing by Synthesis” or SBS. SBS is an optical technique which is very accurate but very slow due to its limitations on DNA read length. Also, the material cost of SBS is high since it requires several expensive reagents to process the DNA sample. In recent years, research in DNA sequencing has been increasingly focused on using disposable microchips to sequence DNA electrically instead of optically. In 2014, Oxford Nanopore Technologies (ONT) commercialized a compact sequencing system based on nanopores. When compared to SBS, the nanopore technology is significantly more portable and cost-effective, although its sequencing accuracy is not as high. ONT is currently recognized as the most successful company in producing nanopore based DNA sequencing systems.

The key element of the ONT system is a disposable microchip embedded with nanopores. In the sequencing process, DNA strands are pulled through the nanopores by electrophoresis and changes of the ionic current through the nanopores are used to determine the base sequence of the DNA strands. ONT's nanopores are fabricated from proprietary protein molecules which have a very short shelf life. In addition, the nanopores must work in tandem with enzyme molecules for the sequencing process to function properly. Recently, researchers have explored using solid-state materials such as silicon to manufacture enzyme-free nanopores. However, this approach has its own limitations. For example, it is difficult to stabilize a DNA strand as it travels through a solid-state nanopore, leading to a large background noise in the sequencing signal. Also, the surface area around a solid-state nanopore is very limited, thus preventing the inclusion of sophisticated sensors needed to accurately resolve the base sequence from the sequencing signal.

It would therefore be desirable to develop a DNA sequencing system, method, and device that overcome these drawbacks of the prior art systems and methods.

### BRIEF SUMMARY OF THE INVENTION

In one embodiment, the present invention is directed to a method of sequencing DNA. A single-stranded DNA molecule is wrapped around a carbon nanotube to form a DNA/carbon nanotube hybrid structure. The DNA/carbon nanotube hybrid structure is then moved across a nanofluidic chip from an inlet end to an outlet end. The nanofluidic chip includes a plurality of graphene sensors, a nanochannel extending across the graphene sensors, and opposing electrodes. A power source applies a constant voltage to the graphene sensors between the opposing electrodes. The resulting electrical current through the graphene sensors between the opposing electrodes is measured as the DNA/carbon nanotube hybrid structure moves across the graphene sensors. The composition of the bases of the single-stranded DNA molecule is determined based on the measured electrical current.

In one embodiment, the present invention is directed to DNA sequencing system, including: a single-stranded DNA molecule wrapped around a carbon nanotube to form a DNA/carbon nanotube hybrid structure; a nanofluidic chip that includes a nanochannel and a plurality of graphene sensors; opposing electrodes; a power source; and an ammeter. The DNA/carbon nanotube hybrid structure is moveable from an inlet end to an outlet end of the nanochannel. The nanochannel extends across the plurality of graphene sensors. The power source applyies constant voltage through the graphene sensors between the opposing electrodes, and the ammeter measures an electrical current through the graphene sensors between the opposing electrodes as the DNA/carbon nanotube hybrid structure moves across the plurality of graphene sensors.

In one embodiment, the present invention is a new DNA sequencing method that uses a novel nanofluidic device coupled with advanced DNA sample preprocessing to overcome the shortcomings of the solid-state nanopores. The DNA sample preprocessing reduces motion-induced background noise in the sequencing signal and the nanofluidic device maximizes the spatial resolution of the signal. With these improvements, the invention will reduce the cost and increase the accuracy of DNA sequencing.

In one embodiment, the sequencing system of the present invention uses a solid-state nanochannel for sequencing that has a much longer shelf life than the protein nanopores used by ONT. The sequencing signal from the sequencing system of the present invention is more stable than the ONT devices. The invention uses DNA-carbon nanotubes (CNT) hybrid structures in the sequencing process. CNTs are hexagonal rings of carbon molecules that form a sheet. A single-walled carbon nanotube (swCNT) is approximately 1 nm in diameter. swCNTs are used as anchors for single-stranded DNA (ssDNA) in the DNA sequencing. A high-efficiency binding protocol was developed to wrap ssDNA of three different lengths (15, 30, and 50 base pairs) around swCNT. As discussed below, results were characterized by Atomic Force Microscopy (AFM) to determine the binding efficiency and wrapping patterns.

These and other features, objects and advantages of the present invention will become better understood from a consideration of the following detailed description of the preferred embodiments and appended claim in conjunction with the drawings as described following:

### BRIEF DESCRIPTION OF THE DRAWINGS

FIGS. 1A-1C show schematics of the present invention. FIG. 1A is a schematic of the DNA/carbon nanotube hybrid structure formed by wrapping a single-stranded DNA around a single-walled carbon nanotube. FIG. 1B is a schematic showing the translocation of a DNA/carbon nanotube hybrid structure through a nanochannel integrated with multiple graphene sensors. FIG. 1C shows a schematic of the hydrophilic bases of ssDNA wrapping around a single-walled carbon nanotube.

FIGS. 2A-2C show the design and fabrication of graphene sequencing sensors. FIG. 2A shows the sensor layout created by LayoutEditor. FIG. 2B shows the AFM nanolithography graphical interface after the sensor layout was imported into the AFM. FIG. 2C shows three identical graphene sensors patterned by AFM nanolithography.

FIGS. 3A-3D show AFM images of ssDNA/swCNT hybrid structures. FIG. 3A is a typical 5 um×5 um image showing multiple hybrid structures. FIG. 3B shows a Poly (T)-15/swCNT. FIG. 3C shows a Poly (T)-30/swCNT. FIG. 3D shows a Poly (T)-50/swCNT.

FIG. 4A is an AFM scan of Poly(T)-50 wrapped around swCNT. FIG. 4B is a height graph across the length of the swCNT and ssDNA complex.

FIG. 5A is an AFM scan of Poly(T)-50 wrapped around swCNT. FIG. 5B is a pitch graph across the width of the swCNT and ssDNA complex.

FIGS. 6A-6D show AFM images of ssDNA/swCNT hybrid structures. FIG. 6A is a global image of Poly(T)-15 ssDNA wrapped around swCNT on a scale of 10×10 um. FIG. 6B is an image of swCNT and ssDNA on a scale of 5×5 um. FIG. 6C is an image of swCNT and ssDNA on a scale of 5×5 um. FIG. 6D is an image of a single nanotube with ssDNA wrapped on a scale of 1.75×1.75 um.

FIGS. 7A-7D show AFM images of ssDNA/swCNT hybrid structures. FIG. 7A is a global image of Poly(T)-30 ssDNA wrapped around swCNT on a scale of 5×5 um. FIG. 7B is an image of swCNT and ssDNA on a scale of 1.8×1.8 um. FIG. 7C is an image of swCNT and ssDNA on a scale of 1.1×1.1 um. FIG. 7D is an image of a single nanotube with ssDNA wrapped on a scale of 0.45×0.45 um.

FIGS. 8A-8D show AFM images of ssDNA/swCNT hybrid structures. FIG. 8A is a global image of Poly(T)-50 ssDNA wrapped around swCNT on a scale of 10×10 um. FIG. 8B is an image of swCNT and ssDNA on a scale of 5×5 um. FIG. 8C is an image of swCNT and ssDNA on a scale of 1.5×1.5 um. FIG. 8D is an image of a single nanotube with ssDNA wrapped on a scale of 0.5×0.5 um.

FIG. 9 shows the patterning of a graphene nanoribbon (GNR) on a silicon microelectrode chip using Local Anodic Oxidation (LAO). The cutting path of monolayer graphene is indicated by dashed lines as shown in the left image. The graphene nanoribbon cut and isolation within the microribbon connecting corresponding electrodes is illustrated in the left image. The AFM image of a typical completed graphene nanoribbon is shown in the right image. The AFM image of an about 150 nm wide patterned graphene nanoribbon is shown in the right image.

FIGS. 10A-10B show a completed microfluidic system embedded with a GNR sequencing sensor. FIG. 10A shows a completed microfluidic system. One well in the PDMS channel is connected to serve as a sample inlet, and the other is left open to allow unrestricted drainage. FIG. 10B show a closeup view of the PDMS aligned bonded to a silicon chip. The PDMS microchannel can be seen centered and parallel to the solder pads.

FIG. 11 shows the experimental setup to validate the performance of the GNR sequencing sensor of the present invention.

FIG. 12 shows four separate electrical current traces (time periods) of the GNR as the hybrid structures passed over the GNR. Time Period 1 is at the top, Time Period 2 is directly below Time Period 1, Time Period 3 is directly below Time Period 2, and Time Period 4 is at the bottom.

### DETAILED DESCRIPTION OF THE INVENTION

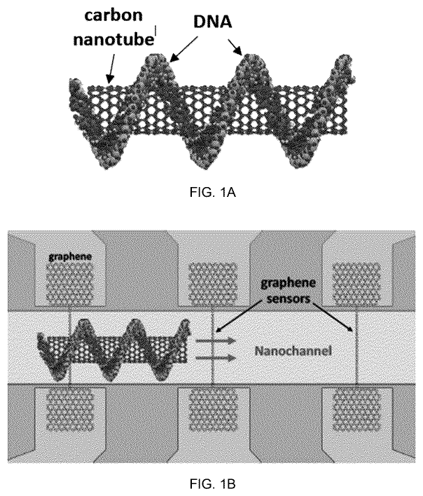

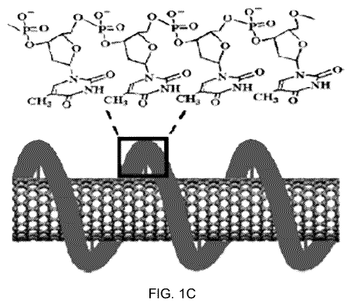

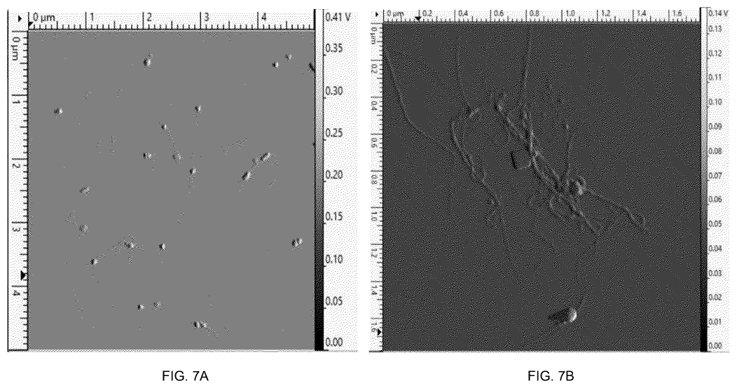

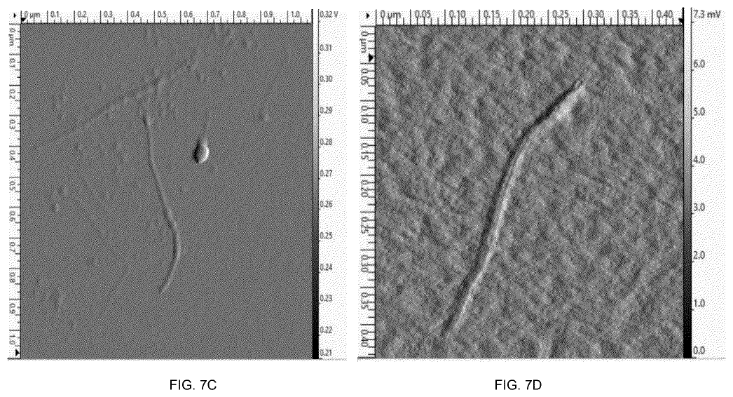

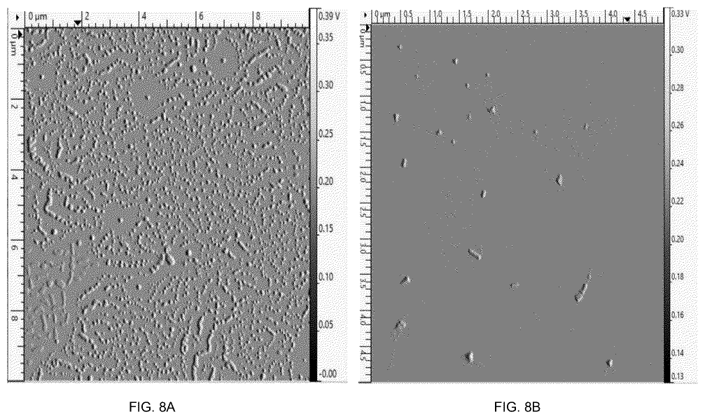

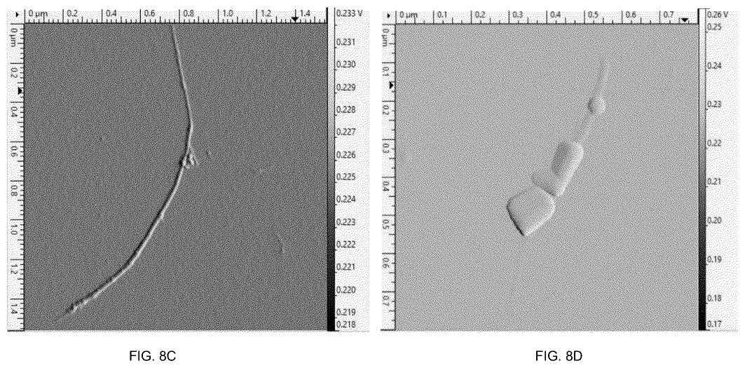

With reference to FIGS. 1A-12, the preferred embodiments of the portable DNA sequencing system utilizing graphene sensors and DNA/CNT hybrid structures may be described. FIGS. 1A-1B demonstrate the schematics of the present invention. The DNA sequencing process begins with a DNA sample preprocessing step where single-stranded DNA (ssDNA) are wrapped around single-walled carbon nanotubes (swCNT) to form “swDNA-swCNT” hybrid structures. Next, the hybrid structures are translocated through a nanofluidic device where the DNA bases on the nanotubes are characterized by multiple graphene sensors in a nanochannel through pi stacking based interaction. The tight ssDNA-to-swCNT binding significantly enhances the structural stability of the DNA bases as they travel through the nanochannel, leading to a significant reduction in the background noise of the sequencing signal. After the hybrid structures move past the graphene sensors, they are collected in a downstream reservoir and disposed of.

In one embodiment, the present sequencing system utilizes a nanochannel instead of a nanopore to sequence DNA. From a structural design standpoint, a nanochannel carries two distinct advantages when compared to a nanopore: (1) a large surface area is available for placing multiple sequencing sensors, and (2) complex hybrid structures formed by DNA and novel nanomaterials can be characterized with high spatial resolution. These two advantages lead to more flexibility in the design of the sequencing sensors and the sample preprocessing protocol.

The present sequencing system is primarily designed for DNA sequencing. However, it is very likely that the same approach can be applied to RNA sequencing. RNA is a highly unstable biomolecule and the nanotube wrapping technique might be a feasible way to stabilize the RNA molecule for accurate sequencing in a nanochannel.

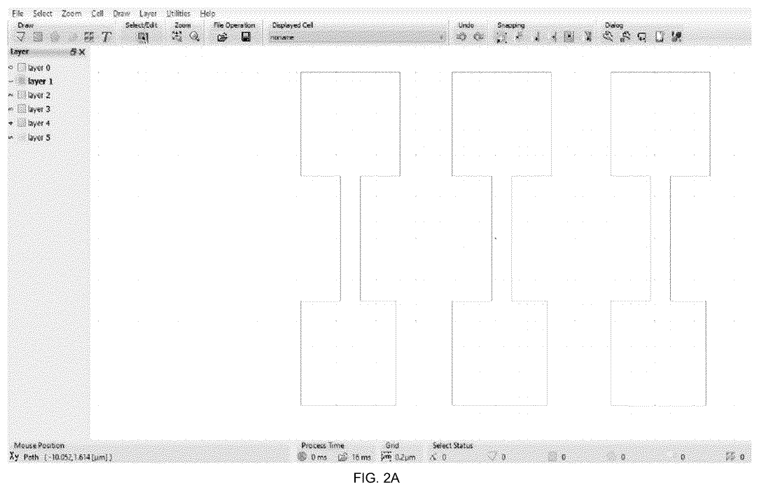

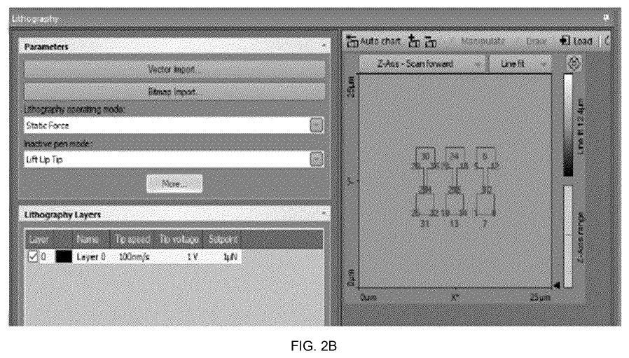

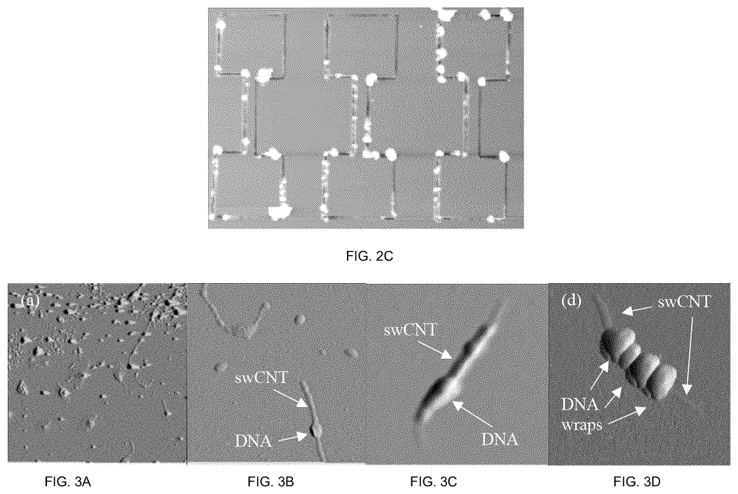

Graphene Sequencing Sensor—The graphene sensors were fabricated using atomic force microscopy (AFM) based nanolithography in a single manufacturing process. AFM nanolithography is a robotic machining technique where a sharp AFM probe is used to pattern a surface material such as graphene with an accuracy at the nanometer level. Graphene is an allotrope of carbon consisting of a single layer of carbon atoms. It is particularly suitable for sensing DNA since they interact strongly through pi-stacking. The previous work of the inventors has indicated that DNA bases alter the electrical conductivity of a graphene sensor as they glance over the graphene surface. FIGS. 2A-2C illustrate the design and fabrication of three identical graphene sensors in a single manufacturing process. First, LayoutEditor, a design software for IC fabrication, was used to define the layout and dimensions of the sensors (FIG. 2A). Next, the design layout was ported into the nanolithography software of the AFM (CoreAFM by Nanosurf, Switzerland) where a machining “path” of the AFM probe was generated either by manually drawing the line segments corresponding to the sensor layout or by importing a 2D vector file into the coreAFM software to dictate the machining path (FIG. 2B). Lastly, the AFM probe mapped out the surface profile of the graphene and then machined the graphene following the predesigned path. FIG. 2C demonstrates the completed graphene sensors.

DNA Sample Preprocessing-A binding protocol to wrap individual ssDNA around individual swCNT was developed. Three different ssDNA lengths were tested: Poly(T)-15, Poly(T)-30, and Poly(T)-50. All three were purchased from Integrated DNA Technologies. The swCNT (purity >95%) were purchased from Sigma-Aldrich. The average diameter of the swCNT is 0.6 nm. The key steps of the binding protocol are as followed. The stock DNA was first diluted by 100 mL of TE buffer to create a 1 mg/mL ssDNA solution. Then, 2 mL of 0.1M NaCl solution and 0.7 mg of swCNT were added to 200 uL of the ssDNA solution. The mixture was sonicated for 3-5 min in an ice bath. The sample was divided into two 1.5 mL centrifuge tubes (about 1100 uL per centrifuge tube). Afterwards, the mixture was centrifuged at 13,000 g for 20 min. The supernatant was extracted and placed in a separate 1.5 mL centrifuge tube and resuspended in deionized (DI) water. Ten uL of the suspension was then placed on a mica disk and allowed to air dry. Finally, 10 uL of the suspension was imaged by an AFM.

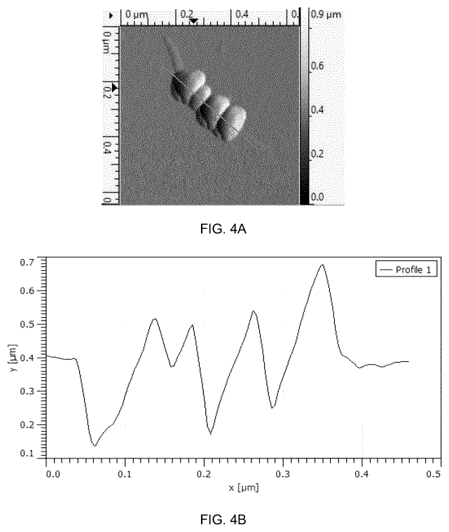

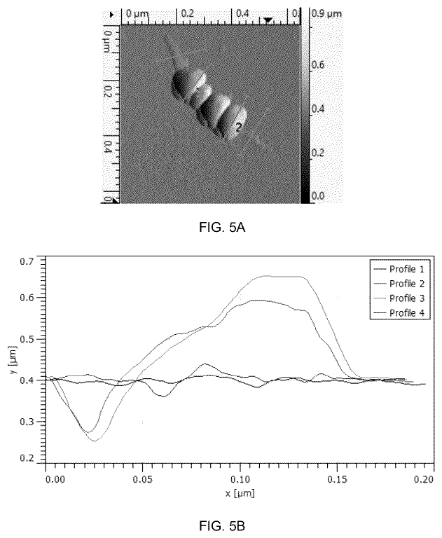

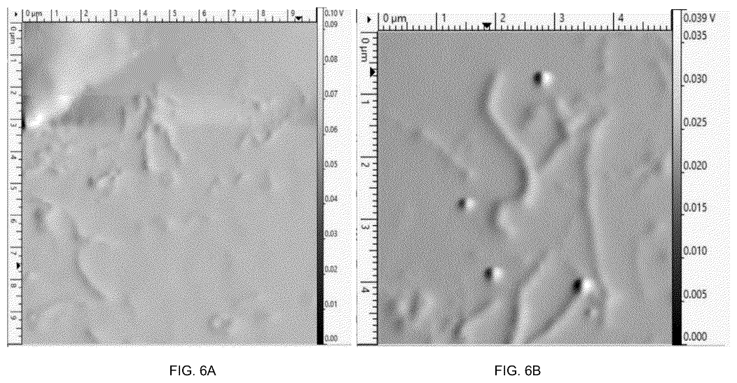

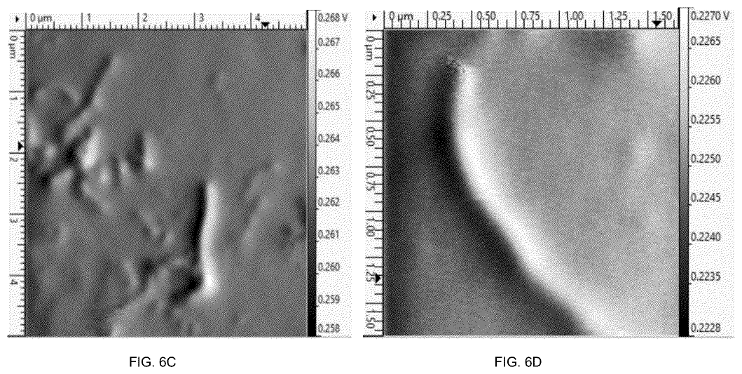

The results shown in FIGS. 3A-3D indicate successful ssDNA wrapping for all three DNA samples with the longest DNA (Poly(T)-50) showing the most identifiable wrapping behavior (FIG. 3D). The pitch and width of the DNA wraps are very uniform, suggesting a high consistency and repeatability when the DNA is sequenced, as shown in FIGS. 4B and 5B. Additional results are shown in FIGS. 6A-8D. These images indicate successful wrapping of ssDNA with swCNT. The images contain unequal wrapping patterns indicated by bulging on branched regions as well as carbon nanotubes that are attracted to one another through intermolecular forces. Extra molecules such as salt can be seen as small spheres on the Poly(T)-15 structures. Global images indicate the wrapping frequencies while local images indicate the wrapping patterns.

This study illustrates that a binding protocol was successfully developed to wrap ssDNA around swCNT, the dispersion rate and binding efficiency were determined to be highest for Poly(T)-50, and the wrapping height and pitch of Poly(T)-50 were determined to be approximately 0.12 um and 0.2 um, respectively.

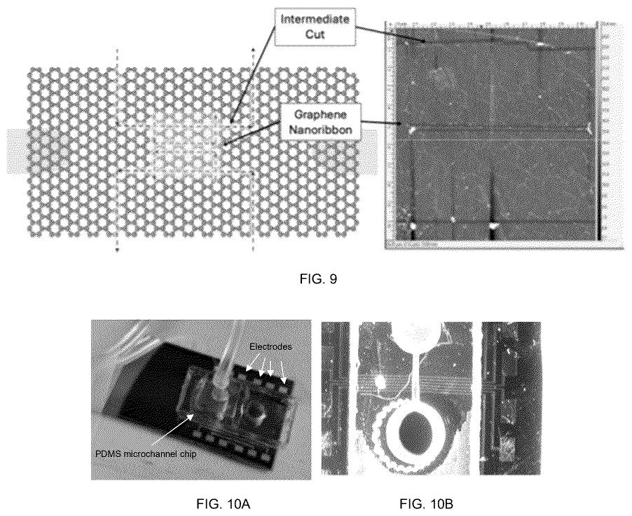

Performance Validation of the Graphene Sequencing Sensor: An integrated microfluidic system was designed, fabricated, and applied to the validation of the graphene nanoribbon (GNR) sequencing sensor. A microchannel instead of a nanochannel was used in the validation experiments due to ease of fabrication. FIG. 9 demonstrates the patterning of a GNR on a silicon microelectrode chip using Local Anodic Oxidation (LAO). After the GNR was patterned, the microfluidic system was completed by bonding a polydimethylsiloxane (PDMS) microchannel over the GNR. FIGS. 10A-10B demonstrates a completed microfluidic system embedded with a GNR sequencing sensor. As indicated in FIG. 10A, the electrodes are located outside the microchannel chip to allow electrical contacts to the graphene sensors.

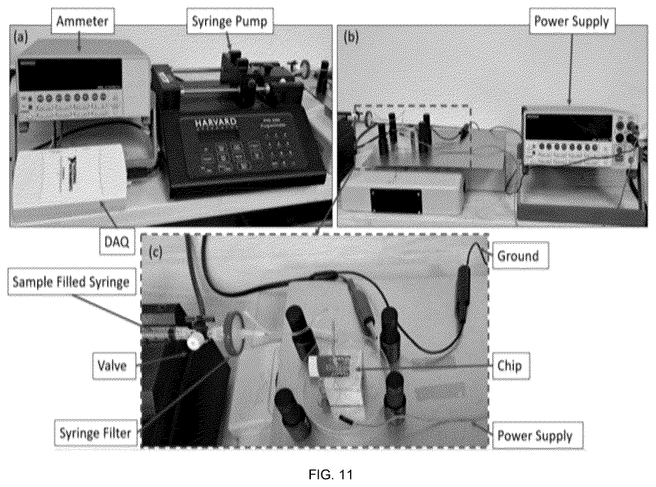

Performance validation of the GNR sequencing sensor was conducted by passing 50 bp-long single-stranded DNA sequence A₁₇C₁₆A₁₇ (ACA) wrapped carbon nanotube hybrid structures over the GNR. In this process, the hybrid structures suspended in deionized water were pumped through the PDMS microchannel using a syringe pump and the resultant change in the conductivity of the GNR was recorded. FIG. 11 demonstrates the experimental setup of the validation experiment, including an ammeter, DAQ, and syringe pump (top left), power supply (top right), and sample filled syringe, valve, syringe filter, ground, power supply, and chip (bottom). The syringe pump delivers a hybrid structure suspension sample loaded in a syringe to the PDMS microchannel at a preset flow rate. The valve removes any air bubbles from the suspension and the syringe filter removes undesirable hybrid structure ‘clumps’ from the suspension. The power supply provides a predetermined DC voltage to the graphene sensors through the electrodes and the ammeter measures the electrical current through the graphene sensors. The data acquisition (DAQ) device converts the analogue current measured by the ammeter to a digital signal which is then recorded and analyzed by a PC. The recorded digital signal can be used to train an Al software to determine the DNA base sequence from the electrical current.

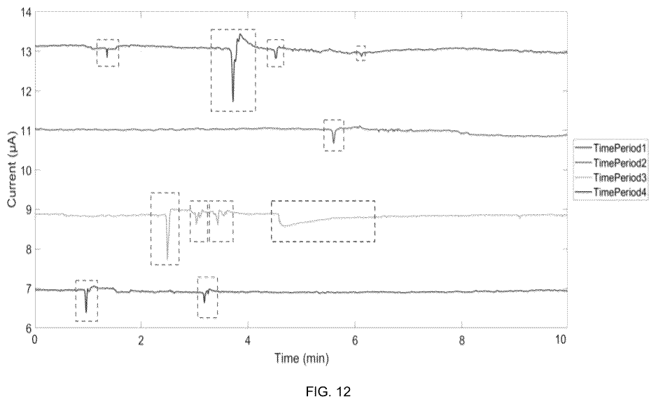

FIG. 12 demonstrates four separate electrical current traces (time periods) of the GNR as the hybrid structures passed over the GNR. The figure demonstrates the presence of distinct “events” where the hybrid structures reduced the electrical conductivity of the GNR. This result is consistent with the hypothesis that the interaction between DNA and GNR is pi stacking in nature. The four traces of a biased GNR recorded during the translocation of ACA wrapped carbon nanotube hybrid structures are shown in FIG. 12. Each DNA induced ‘event’ is marked by a dashed box. The dashed box on the right of the second trace from the bottom marks the passage of an air bubble, which looks very different from that of a DNA induced “event.”

### References

1. Mardis, E. R. Next-generation DNA sequencing methods. Annu. Rev. Genomics Hum. Genet. 9, 387-402 (2008).
2. Zwolak, M. & Di Ventra, M. Colloquium: Physical approaches to DNA sequencing and detection. Rev. Mod. Phys. 80, 141-165 (2008).
3. Rhoads, A. & Au, K. F. PacBio Sequencing and Its Applications. Genomics. Proteomics Bioinformatics 13, 278-289 (2015).
4. Caporaso, J. G., Lauber, C. L., Walters, W. A., Berg-Lyons, D., Huntley, J., Fierer, N., Owens, S. M., Betley, J., Fraser, L., Bauer, M., Gormley, N., Gilbert, J. A., Smith, G. & Knight, R. Ultra-high-throughput microbial community analysis on the Illumina HiSeq and MiSeq platforms. ISME J. 6, 1621-1624 (2012).
5. Sboner, A., Mu, X., Greenbaum, D., Auerbach, R. K. & Gerstein, M. B. The real cost of sequencing: higher than you think! Genome Biol. 12, 125 (2011).
6. Check Hayden, E. Is the $1,000 genome for real? Nature (2014). doi:10.1038/nature.2014.14530
7. Rusk, N. Cheap third-generation sequencing. Nat. Methods 6, 244-244 (2009).
8. Clarke, J., Wu, H.-C. H., Jayasinghe, L., Patel, A., Reid, S. & Bayley, H. Continuous base identification for single-molecule nanopore DNA sequencing. Nat. Nanotechnol. 4, 265-70 (2009).
9. Wanunu, M. Nanopores: A journey towards DNA sequencing. Phys. Life Rev. 9, 125-58 (2012).
10. Deamer, D., Akeson, M. & Branton, D. Three decades of nanopore sequencing. Nat. Biotechnol. 34, 518-524 (2016).

The present invention has been described with reference to certain preferred and alternative embodiments that are intended to be exemplary only and not limiting to the full scope of the present invention as set forth in the appended claims.

We claim:

1. A method of sequencing DNA, comprising the steps of:
wrapping a single-stranded DNA molecule around a carbon nanotube to form a DNA/carbon nanotube hybrid structure, wherein said single-stranded DNA molecule comprises at least one base;
moving said DNA/carbon nanotube hybrid structure from an inlet end of a nanofluidic chip to an outlet end of said nanofluidic chip, wherein said nanofluidic chip comprises a plurality of graphene sensors and a nanochannel extending across said plurality of graphene sensors, wherein said plurality of graphene sensors are positioned between opposing electrodes, wherein a power source applies a constant voltage to said plurality of graphene sensors between said opposing electrodes;
measuring an electrical current through said plurality of graphene sensors as said DNA/carbon nanotube hybrid structure moves across said plurality of graphene sensors; and
determining a composition of said at least one base of said single-stranded DNA molecule based on the measured electrical current.

2. The method of claim 1, wherein said carbon nanotube is single-walled.

3. The method of claim 1, wherein a graphene nanoribbon comprises said plurality of graphene sensors.

4. A DNA sequencing system, comprising:
a single-stranded DNA molecule wrapped around a carbon nanotube, thereby forming a DNA/carbon nanotube hybrid structure, wherein said single-stranded DNA molecule comprises at least one base;
a nanofluidic chip on which said DNA/carbon nanotube hybrid structure is moveable from an inlet end to an outlet end of said nanofluidic chip, wherein said nanofluidic chip comprises a plurality of graphene sensors and a nanochannel extending across said plurality of graphene sensors, wherein said plurality of graphene sensors are positioned between opposing electrodes;
a power source configured to apply a constant voltage to said plurality of graphene sensors between said opposing electrodes; and
an ammeter configured to measure an electrical current through the graphene sensors between said opposing electrodes as said DNA/carbon nanotube hybrid structure moves across said plurality of graphene sensors.

5. The system of claim 4, wherein said carbon nanotube is single-walled.

6. The system of claim 4, wherein a graphene nanoribbon comprises said plurality of graphene sensors.
